(19) United States
(12) Patent Application Publication
(10) Pub. No.: US 2012/0215023 A1
(43) Pub. Date: Aug. 23, 2012

(54) Method for Preparative Fragmenting Using an Inductively Heated Heating Medium

(76) Inventors: Carsten Friese, Duesseldorf (DE); Andreas Kirschning, Celle (DE); Sascha Volkan Ceylan, Hannover (DE); Juergen Wichelhaus, Wuppertal (DE)

(21) Appl. No.: 13/400,732

(22) Filed: Feb. 21, 2012

Related U.S. Application Data
(63) Continuation of application No. PCT/EP2010/059233, filed on Jun. 29, 2010.

(30) Foreign Application Priority Data
Aug. 26, 2009 (DE) 102009028856.2-45

(51) Int. Cl. C07C 253/30; C07C 4/04; C07C 51/38

(52) U.S. Cl. 558/442; 562/598; 558/443; 585/648

(57) ABSTRACT

A method for performing a chemical reaction to produce a target compound by heating a reaction medium containing a reactant in a reactor, the reaction medium being brought into contact with a solid heating medium that can be heated by electromagnetic induction and that is located inside the reactor and surrounded by the reaction medium and the heating medium being heated by electromagnetic induction with the aid of an inductor, causing the target compound to form, and the target compound being separated from the heating medium, wherein the target compound has a lower molar mass than the reactant and at least one covalent bond of the reactant is cleaved in order to produce said target compound from the reactant.

### CROSS-REFERENCE TO RELATED APPLICATIONS

This application is a continuation of International Patent Application No. PCT/EP2010/059233 filed Jun. 29, 2010, which claims priority to German Patent Application No. 102009028856.2 filed Aug. 25, 2009, the contents of both of which are incorporated herein by reference.

The present invention lies within the field of chemical synthesis and relates to a method for performing a fragmentation reaction or a pyrolysis using an inductively heated heating medium.

Various methods are known for heating the reactants in order to perform chemical reactions that can be induced thermally. The most widespread of these is heating by means of heat conduction. Here the reactants are located in a reactor, and either the walls of the reactor itself are heated or heat-transmitting elements such as heating coils or heat exchanger tubes or plates, for example, are built into the reactor. This method is comparatively sluggish, such that on the one hand heating of the reactants occurs slowly and on the other the input of heat cannot be stopped quickly or even replaced by cooling. An alternative consists of heating the reactants by radiating microwaves into the reactants themselves or into a medium containing the reactants. Microwave generators present a considerable risk to safety, however, as they use complex equipment and there is a danger of an escape of radiation.

By contrast, the present invention provides a method in which the reaction medium is heated by bringing it into contact with a heating medium that can be heated by electromagnetic induction, said heating medium being heated “from outside” by electromagnetic induction with the aid of an inductor.

The inductive heating method has been used in industry for some time. The commonest applications are melting, hardening, sintering and the heat treatment of alloys. However, processes such as gluing, shrinking or joining of components are also known applications of this heating method.

Methods for isolating and analyzing biomolecules are known from the German patent application DE 198 00 294, wherein said biomolecules are bound to the surface of magnetic particles that can be heated by induction. This document states:

“The principle of operation consists in adsorptively or covalently binding biomolecules to the surface of a functional polymer matrix, in which the inductively heatable magnetic colloids or finely dispersed magnetic particles are encapsulated, said biomolecules being capable of binding analytes such as e.g. DNA/RNA sequences, antibodies, antigens, proteins, cells, bacteria, viruses or fungal spores according to the complementary affinity principle. Once the analytes have been bound to the matrix the magnetic particles can be heated in a high-frequency magnetic alternating field to temperatures of preferably 40 to 120° C. that are relevant for analysis, diagnostics and therapy.” This document goes on to cover the technical design of coil systems and high-frequency generators that can be used in this method. The cited document thus describes the use of inductively heatable particles in the analysis of complex biological systems or biomolecules.

DE 10 2005 051637 describes a reactor system with a microstructured reactor and methods for performing a chemical reaction in such a reactor. The reactor as such is heated by electromagnetic induction. Heat is transferred into the reaction medium via the heated reactor walls. This limits the size of the surface area that is available for heating the reaction medium. It is also necessary to heat parts of the reactor that are not in direct contact with the reaction medium.

U.S. Pat. No. 5,110,996 describes the production of vinylidene fluoride by reacting dichlorodifluoromethane with methane in the gas phase in a heated reactor. The reactor is filled with a non-metallic filler. The reaction chamber containing this filler is encased by a metallic jacket, which is heated from outside by electromagnetic induction. Thus the reaction chamber itself is heated from outside, causing the filler likewise to be heated at the same time by heat radiation and/or heat conduction. Direct heating by electromagnetic induction of the filler around which the reactants flow does not occur even if this filler is electrically conductive, as the metallic reactor wall shields the electromagnetic fields from the induction coil.

WO 95/21126 discloses a method for producing hydrogen cyanide in the gas phase from ammonia and a hydrocarbon using a metallic catalyst. The catalyst is located inside the reaction chamber, such that the reactants flow around it. It is heated from outside by electromagnetic induction at a frequency of 0.5 to 30 MHz, in other words by an alternating field in the high-frequency range. This document quotes the aforementioned document U.S. Pat. No. 5,110,996, with the comment that inductive heating is conventionally performed in the frequency range from approximately 0.1 to 0.2 MHz. This statement is not however included in the cited U.S. Pat. No. 5,110,996, so it is unclear to what it relates.

WO 00/38831 addresses controlled adsorption and desorption processes, in which the temperature of the adsorbent material is controlled by electromagnetic induction.

It is known from the journal article “Induktives Heizen in der organischen Synthese . . . ” by S. Ceylan, C. Friese, Ch. Lammel, K. Mazac and A. Kirschning, Angew. Chem 2008 (129), pp. 9083-9086, Angew. Chem Int. Ed. 2008 (47), pp. 8950-8953, that chemical reactions can be performed by heating a heating medium using electromagnetic induction. The German patent application DE 102007059967 and the international application WO 2009/074373 have since been published, which describe the subject-matter of the cited journal article in more detail. A number of reactions are mentioned there by way of example.

The present invention provides a method for performing a chemical reaction to produce a target compound by heating a reaction medium containing a reactant in a reactor, the reaction medium being brought into contact with a solid heating medium that can be heated by electromagnetic induction and that is located inside the reactor and surrounded by the reaction medium and the heating medium being heated by electromagnetic induction with the aid of an inductor, causing the target compound to form, and the target compound being separated from the heating medium, wherein the target compound has a lower molar mass than the reactant and at least one covalent bond of the reactant is cleaved in order to produce said target compound from the reactant. The molar mass of the target compound is preferably at most half as large as that of the reactant.

Such a reaction can be described as a fragmentation or pyrolysis. The reaction can lead to the target compound in a single step, i.e. with cleavage of a single covalent bond (optionally combined with the rearrangement of H atoms). However, the target compound can also be formed in a sequence of two or more chemical reactions via one or more intermediate compounds, wherein at least one reaction step includes the cleavage of a covalent bond. Not only can this bond cleavage be accompanied by a rearrangement of H atoms, but other inter- or intramolecular rearrangements can also occur until the target compound is formed.

The covalent bond to be cleaved can in particular be a C—C bond, a C—O bond, a C—N bond, a C—Se bond or a C—S bond.

The “target compound” is understood to be any compound obtained in macroscopic amounts as a result of the method according to the invention as an isolated substance, as a component of a mixture of substances or as a solution in a solvent. Macroscopic amounts are understood to be amounts of at least 100 mg, preferably at least 1 g and in particular at least 100 g per working day. The target compound can also consist of a mixture of different molecules, as is the case for example with the pyrolysis of oils. Therefore the method is not used analytically, wherein relatively large molecules are fragmented in order to establish their identity or structure, as is known from the German patent application DE 198 00 294.

The reaction in which a covalent bond is cleaved is thus started and optionally maintained by heating a reaction medium containing the reactant. This includes the possibility that the reaction medium, a liquid for example, is itself the reactant. Alternatively the reactant can be dissolved or dispersed in the reaction medium.

The solid heating medium is surrounded by the reaction medium. This can mean that the solid heating medium, with the exclusion of possible edge zones, is located within the reaction medium, for example if the heating medium is in the form of particles, chips, wires, meshes, wool, fillers, etc. However, it can also mean that the reaction medium flows through a large number of voids in the heating medium if the latter consists for example of one or more membranes, a bundle of tubes, a rolled metal foil, frits, porous fillers or a foam. In this case too the heating medium is substantially surrounded by the reaction medium, as the majority of its surface area (90% or more) is in contact with the reaction medium. By contrast, in a reactor whose outer wall is heated by electromagnetic induction (in accordance with the cited document U.S. Pat. No. 5,110,996 for example), only the inner surface of the reactor is in contact with the reaction medium.

The wall of the reactor is made from a material that does not shield or absorb the electromagnetic alternating field generated by the inductor and so does not heat up itself. Metals are therefore unsuitable. It can consist for example of plastic, glass or ceramic (such as for example silicon carbide or silicon nitride). The latter is suitable in particular for reactions at high temperature (500 to 600° C.) and/or under pressure.

The processing mode described above has the advantage that the heat energy for triggering and/or performing the chemical reaction is not introduced into the reaction medium via surfaces such as for example the reactor walls, heating coils, heat exchanger plates or similar but rather it is generated directly in the body of the reactor. The ratio of heated surface area to volume of the reaction medium can be substantially greater than in the case of heating via heat-transferring surfaces, as is also the case for example in accordance with DE 10 2005 051637 cited in the introduction. In addition, the efficiency of electric current to heating capacity is improved. By switching on the inductor, heat can be generated in the entire solid heating medium, which is in contact with the reaction medium via a very large surface area. When the inductor is switched off, the further introduction of heat is very quickly stopped. This allows a very targeted reaction control.

Once the target compound has been formed, it is separated from the heating medium. Ideally the target compound is isolated in pure form, in other words without solvent and with no more than the usual impurities. However, the target compound can also be separated from the heating medium in a mixture with reactants or as a solution in the reaction medium and only subsequently isolated by further processing or transferred to a different solvent if desired. The method can therefore be used for the preparative production of the target compound to enable it to be used further.

By contrast, there are methods in which a chemical reaction is likewise started by electromagnetic induction of a heating medium but this reaction is not used to produce a target compound that is separated from the heating medium after the end of the reaction. One example of this is the curing of resin systems, in which the curing reaction is started at particles that are dispersed in the resin system and are heated by electromagnetic induction. These particles remain in the cured resin system and no defined target compound is isolated. The same applies to the converse situation in which an adhesive bond is dissolved again by the inductive heating of particles in the adhesive matrix. A chemical reaction can take place here, but no target compounds are isolated.

The heating medium consists of an electrically conductive and/or magnetizable material that heats up under the influence of an electromagnetic alternating field. It is preferably selected from materials having a very large surface area in comparison to their volume. For example, the heating medium can be selected from electrically conductive chips, wires, meshes, wool, membranes, porous frits, tube bundles (comprising three or more tubes), rolled metal foil, foams, fillers such as for example granules or balls, Raschig rings and in particular from particles, which preferably have an average diameter of no more than 1 mm. For example, metallic mixing elements such as are used for static mixers can be used as the heating medium. In order to be capable of being heated by electromagnetic induction, the heating medium is electrically conductive, for example metallic (wherein it can be diamagnetic), or it has an increased interaction with a magnetic field in comparison to diamagnetism and is in particular ferromagnetic, ferrimagnetic, paramagnetic or superparamagnetic. It makes no difference whether the heating medium is organic or inorganic in nature or whether it contains both inorganic and organic components.

In a preferred embodiment the heating medium is selected from particles of electrically conductive and/or magnetizable solids, wherein the particles have an average particle size in the range from 1 nm to 1000 nm, in particular from 10 nm to 500 nm, such as for instance from 20 nm to 250 nm.

Within the context of the present invention the term “average particle size” is preferably understood to mean the volume-average D50 particle diameter. The volume-average D50 particle diameter is the point in the particle size distribution at which 50 vol. % of the particles have a smaller diameter and 50 vol. % of the particles have a larger diameter.

The average particle size and if necessary also the particle size distribution can be determined by light scattering for example, using for instance a Malvern Mastersizer 2000 from Malvern Instruments Ltd., with which the volume-average D50 particle diameter can be determined using Mie theory.

Magnetic particles, for example ferromagnetic or superparamagnetic particles, are preferably chosen that have as low as possible a remanence or residual magnetization. This has the advantage that the particles do not adhere to one another. The magnetic particles can be present for example in the form of “ferrofluids”, in other words liquids in which ferromagnetic particles are dispersed in the nano-size scale. The liquid phase of the ferrofluid can then serve as the reaction medium.

Magnetizable particles, in particular ferromagnetic particles, having the desired properties are known in the prior art and are commercially available. The commercially available ferrofluids are mentioned by way of example. Examples of the production of magnetic nanoparticles that can be used in the context of the method according to the invention can be taken from the article by Lu, Salabas and Schüth: “Magnetische nano-Partikel: Synthese, Stabilisierung, Funktionalisierung and Anwendung”, Angew. Chem. 2007, 119, pages 1242 to 1266.

Suitable magnetic nanoparticles are known with differing compositions and phases. Examples that can be cited include: pure metals such as Fe, Co and Ni, oxides such as Fe₃O₄ and gamma-Fe₂O₃, spinel-like ferromagnets such as MgFe₂O₄, MnFe₂O₄ and CoFe₂O₄ and alloys such as CoPt₃ and FePt. The magnetic nanoparticles can have a homogeneous structure or a core-shell structure. In the latter case the core and shell can consist of different ferromagnetic or antiferromagnetic materials. Embodiments are also possible, however, in which at least one magnetizable core, which can be ferromagnetic, antiferromagnetic, paramagnetic or superparamagnetic for example, is surrounded by a non-magnetic material. This material can be an organic polymer, for example. Alternatively the shell consists of an inorganic material such as for example silicic acid or SiO₂. A coating of this type can prevent a chemical interaction between the reaction medium or reactants and the material of the magnetic particles themselves. Furthermore, the shell material can be surface-functionalized without the material of the magnetizable core interacting with the functionalizing species. Several particles of the core material can also be enclosed together in such a shell.

Nanoscale particles of superparamagnetic substances selected from aluminum, cobalt, iron, nickel or alloys thereof, metal oxides of the n-maghemite type (gamma-Fe₂O₃), n-magnetite (Fe₃O₄) or ferrites of the MeFe₂O₄ type, where Me is a divalent metal selected from manganese, copper, zinc, cobalt, nickel, magnesium, calcium or cadmium, can be used for example as the heating medium. These particles preferably have an average particle size of ≦100 nm, preferably ≦51 nm and in particularly preferably ≦30 nm.

A material that is available from Evonik (formerly Degussa) under the name MagSilica® is suitable, for example. In this material iron oxide crystals measuring 5 to 30 nm are embedded in an amorphous silicic acid matrix. Iron oxide-silicon dioxide composite particles that are described in more detail in the German patent application DE 101 40 089 are particularly suitable.

These particles can contain superparamagnetic iron oxide domains having a diameter of 3 to 20 nm. These are understood to be physically separated superparamagnetic areas. The iron oxide can be present in these domains in a uniform modification or in different modifications. A particularly preferred superparamagnetic iron oxide domain is gamma-Fe₂O₃, Fe₃O₄ and mixtures thereof.

The proportion of superparamagnetic iron oxide domains in these particles can be between 1 and 99.6 wt. %. The individual domains are separated from one another and/or surrounded by a non-magnetizable silicon dioxide matrix. The range having a proportion of superparamagnetic domains of >30 wt. % is preferred, particularly preferably >50 wt. %. As the proportion of superparamagnetic areas increases, so too does the achievable magnetic effect of the particles according to the invention. In addition to physically separating the superparamagnetic iron oxide domains, the silicon dioxide matrix also serves to stabilize the oxidation stage of the domains. Thus magnetite for example as the superparamagnetic iron oxide phase is stabilized by a silicon dioxide matrix. These and other properties of these particles that are particularly suitable for the present invention are set out in more detail in DE 101 40 089 and in WO 03/042315.

Furthermore, nanoscale ferrites such as are known for example from WO 03/054102 can be used as the heating medium. These ferrites have a (M^{a}_{1-x-y} M^{b}ₓ Fe^{II}_{y}) Fe^{III}₂O₄ composition, in which
M^{a} is selected from Mn, Co, Ni, Mg, Ca, Cu, Zn, Y and V,
M^{b} is selected from Zn and Cd,
x denotes 0.05 to 0.95, preferably 0.01 to 0.8,
y denotes 0 to 0.95 and
the sum of x and y is at most 1.

The method according to the invention can in principle be performed continuously or batchwise. If the reaction is performed batchwise, the reaction medium and the inductively heated solid heating medium are preferably moved relative to each other during the reaction. If a particulate heating medium is used, this can take place in particular by stirring the reaction medium together with the heating medium or by vortexing the heating medium in the reaction medium. If for example meshes or wool of a threadlike heating medium are used, the reaction vessel containing the reaction medium and the heating medium can be shaken for example.

A preferred embodiment of a reaction performed batchwise consists of placing the reaction medium in a reaction vessel together with particles of the heating medium and moving it with the aid of a moving element located in the reaction medium, the moving element being set up as an inductor by means of which the particles of the heating medium are heated by electromagnetic induction. Thus in this embodiment the inductor is located inside the reaction medium. The moving element can take the form of a stirrer or a vertically moving plunger, for example.

It can additionally be provided that the reactor is externally cooled during the chemical reaction. This is possible in batch operation in particular, if, as stated above, the inductor is immersed in the reaction medium. The introduction of the electromagnetic alternating field into the reactor is then not inhibited by the cooling device.

The reactor can be cooled internally via cooling coils or heat exchangers or preferably externally. Optionally precooled water or a cooling mixture whose temperature is below 0° C. can be used for cooling, for example. Examples of such cooling mixtures are ice/common salt mixtures, methanol/dry ice or liquid nitrogen. Cooling allows a temperature gradient to be established between the reactor wall and the inductively heated heating medium. This is particularly pronounced if a cooling mixture with a temperature well below 0° C. is used, for example methanol/dry ice or liquid nitrogen. The reaction medium that is heated by the inductively heated heating medium is then cooled again externally. The chemical reaction of the reactant then takes place only if it is in contact with the heating medium or is at least in its immediate vicinity. Owing to the movement of the reaction medium relative to the heating medium, the product species formed in the reaction quickly move into the cooler areas of the reaction medium, thus inhibiting their further thermal reaction. In this way if there are several possible reaction pathways for the reactant(s), a desired reaction pathway can be selected kinetically.

In an alternative embodiment the chemical reaction is performed continuously in a continuous-flow reactor that is at least partially filled with the solid heating medium and thus has at least one heating zone that is capable of being heated by electromagnetic induction, the reaction medium flowing continuously through the continuous-flow reactor and the inductor being located outside the reactor. Here the reaction medium flows around the heating medium, if for example the latter is in the form of particles, chips, wires, meshes, wool, fillers, etc. Alternatively, the reaction medium flows through the heating medium through a large number of holes in the heating medium if the latter consists for example of one or more membranes, frits, porous fillers or a foam.

The continuous-flow reactor is preferably designed as a tubular-flow reactor. In this case the inductor can surround the reactor completely or at least partially. The electromagnetic alternating field generated by the inductor is then introduced on all sides or at least from several points into the reactor.

“Continuous” is understood here in the usual way to be a reaction control in which the reaction medium flows through the reactor at least over a period such that a total volume of reaction medium that is large in comparison to the internal volume of the reactor itself has flowed through the reactor before the flow of the reaction medium is stopped. “Large” in this sense means “at least twice as large”. Even such a continuously performed reaction has a beginning and an end of course.

In this continuous processing mode in a continuous-flow reactor it is possible for the reactor to have several heating zones. For example, different heating zones can be heated to differing extents. This can be achieved either by the positioning of different heating media in the continuous-flow reactor or by means of differently configured inductors along the reactor. Different heating conditions can be established in this way for the formation of one or more intermediate compounds and the target compound.

It is also possible to proceed by first preheating the solvent or the reaction medium in a conventional manner before bringing it into contact with the heating medium in order to perform the reaction.

A cooling zone can be provided after the (last) heating zone if desired, for example in the form of a cooling jacket around the reactor.

It can also be provided that after leaving the heating zone the reaction medium is brought into contact with an absorber substance that removes by-products or impurities from the reaction medium. This can for example be a molecular sieve, through which the reaction medium flows after leaving the heating zone. This allows the product to be purified immediately after being produced.

Depending on the speed of the chemical reaction, the product yield can optionally be increased by returning at least part of the reaction medium that has flowed through the solid heating medium to flow through the solid heating medium again. It can be provided for impurities, by-products or the desired main product to be removed from the reaction medium after each passage through the solid heating medium. The various known separating methods are suitable for this purpose, for example absorption on an absorber substance, separation by a membrane method, precipitation by cooling or separation by distillation. A complete reaction of the reactant(s) can ultimately be achieved in this way.

The entire contact time of the reaction medium with the inductively heated heating medium that is conveniently chosen is dependent on the kinetics of the individual chemical reaction. The slower the desired reaction, the longer the chosen contact time should be. This must be adjusted empirically in the individual case. As a reference point, the reaction medium should preferably pass through the continuous-flow reactor one or more times at a rate such that the entire contact time of the reaction medium with the inductively heated heating medium is in the range from approximately 1 second to approximately 2 hours before the target compound is separated off. Shorter contact times are conceivable, but are more difficult to control. Longer contact times can be necessary with particularly slow chemical reactions, but they incrementally reduce the cost-effectiveness of the method.

The method according to the invention is preferably performed in such a way that under the established reaction conditions (in particular temperature and pressure) the reaction medium is present in the reactor as a liquid. Relative to the reactor volume, better volume/time yields are generally possible in this way than is the case with reactions in the gas phase. The reaction can however also take place in the gas phase, but with the advantage of lower volume yields.

It goes without saying that the nature of the heating medium and the design of the inductor must be matched to one another so that the reaction medium can be heated in the desired way. Critical variables here are the output of the inductor expressed in Watts and the frequency of the alternating field generated by the inductor. In principle, the greater the mass of the heating medium to be heated inductively, the higher the chosen output must be. In practice, the achievable output is limited in particular by the possibility of cooling the generator needed to supply the inductor.

Inductors generating an alternating field with a frequency in the range from approximately 1 to approximately 100 kHz, preferably from 10 to 80 kHz and in particular in the range from approximately 10 to approximately 50 kHz, particularly up to 30 kHz, are particularly suitable. Such inductors and the associated generators are available commercially, for example from IFF GmbH in Ismaning (Germany).

The inductive heating is thus preferably performed with an alternating field in the medium-frequency range. In comparison with an excitation at higher frequencies, for example at those in the high-frequency range (frequencies above 0.5, in particular above 1 MHz), this has the advantage that the introduction of energy into the heating medium is more readily controllable. This applies in particular if the reaction medium is present as a liquid under the reaction conditions. In the context of the present invention it is therefore preferable for the reaction medium to be present as a liquid and for inductors to be used that generate an alternating field in the aforementioned medium-frequency range. This allows a cost-effective and readily controllable reaction control.

The following for example can be used as the heating medium:
a) MagSilica® 58/85 from Evonik (formerly Degussa),
b) Manganese ferrite powder from SusTech GmbH, Darmstadt,
c) Bayferrox® 318 M: synthetic alpha-Fe₃O₄ from Harold Scholz & Co. GmbH,
d) Manganese zinc ferrite, surface-coated with oleic acid, ferrite content 51.7 wt. %, SusTech GmbH, Darmstadt,
e) Balls or other moldings, rolled metal sheets, chips, rolled meshes or metal wool,
f) Fe₂O₃, in particular in the form of nanoparticles having a particle size in the range from 20 to 200 nm, or Fe₃O₄, in particular in the form of nanoparticles having a particle size in the range from 20 to 200 nm (both available from DKSH GmbH, Germany),
g) Steel balls, for example ball bearings, preferably having a diameter of at most 1 mm, for example between 0.5 and 1 mm.

In a special embodiment of the method according to the invention the heating medium is ferromagnetic and has a Curie point in the range from approximately 40 to approximately 250° C., which is selected such that the Curie point differs by no more than 20° C., preferably by no more than 10° C., from the selected reaction temperature. This leads to an inherent protection against inadvertent overheating. The heating medium can only be heated by electromagnetic induction up to its Curie point, while at a higher temperature it is no longer heated by the electromagnetic alternating field. Even if the inductor malfunctions, the temperature of the reaction medium can be prevented in this way from rising inadvertently to a value well above the Curie point of the heating medium. If the temperature of the heating medium falls below its Curie point again, it can once again be heated by electromagnetic induction. This leads to a self-regulation of the temperature of the heating medium in the vicinity of the Curie point.

The following reactions for example can be performed by the method according to the invention, the invention not being restricted thereto.
a) Cleavage or pyrolysis of fats or oils (for which temperatures in the range from at least 200° C., in particular at least 300° C., to approximately 600° C. can be necessary),
b) Depolymerization reactions, for example the cleavage of polyolefins or of cyanoacrylate polymers or oligomers into smaller fragments or monomers (which in the case of cyanoacrylates requires temperatures in the range from approximately 140° C. to approximately 200° C.),
c) Retro-cycloadditions (such as retro-Diels-Alder reactions),
d) Grob fragmentations (in their general form):

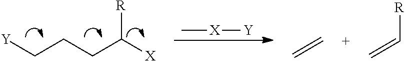

### EXAMPLES

The examples below show reactions that can be performed in a continuous-flow reactor on a laboratory scale by the method according to the invention. The present invention is of course not restricted thereto.

Glass tubes of length 10 cm and of various internal and external diameters were used as tubular-flow reactors. The tubes were provided with screw connections at both ends to allow HPLC attachments and the appropriate hoses to be attached.

The inductor used had the following performance features: inductance: 134 μHenry, number of coil turns: 2·16, cross-sectional area=2.8 mm² (the cross-sectional area is calculated from the number of conductor wires used in the inductor and their diameter). The diameter of the gap to hold the tubular-flow reactors was 12 mm. In all experiments the inductor was operated at a frequency of 25 kHz.

In the experiments performed the specified frequency of 25 kHz was kept constant and the heating controlled only by means of PWM (PWM=switching a square-wave signal on and off at a fixed basic frequency). The PWM is given below in ‰. The induced temperature was measured using a thermocouple and an infrared thermometer. The thermocouple was attached directly behind the reactor in the fluid in order to allow as accurate a measurement as possible. Because of the metallic components of the temperature sensor, however, a minimum gap of 4 cm had to be maintained. A laser infrared thermometer with close-focus lens was used as the second temperature measuring instrument. The measuring dot had a diameter of 1 mm. This method was intended for measuring the surface temperature of the reactor so as to obtain a second measuring point for the temperature determination. In infrared measurement the emission factor of the material is an important constant. It is a measure of heat radiation. An emission factor of 0.85 was used, which corresponds to an average glass.

Decarboxylation Reactions:

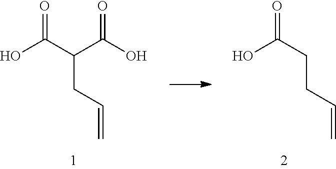

A glass reactor (length 12 cm, internal diameter 8.5 mm) is filled with MagSilica™ and zinc (˜5.5 g) and both ends are closed with cotton wool. An HPLC pump is attached to one end of the reactor, while the other end is connected to a receiver. The reactor is inserted in the inductor and then rinsed with N,N-dimethylformamide (DMF). Then a flow rate of 0.2 ml/min is established and the reactor temperature is set to 135° C. (excitation frequency: 25 kHz, output setting: 550 per mil). Once a constant temperature has been reached a solution of dicarboxylic acid 1 (50 mg, 0.35 mmol) in DMF (5 ml) is pumped through the reactor at 0.2 ml/min. Then a further 15 ml of DMF are pumped through the reactor. The combined organic phases are acidulated with 4 M HCl and extracted repeatedly with ethyl acetate. The combined organic phases are concentrated to low volume over MgSO₄ dried under vacuum. The title compound 2 is obtained with no further purification as a colorless oil (26.7 mg, 0.27 mmol, 76% yield). ¹H-NMR (400 MHz, CDCl₃, for main product): 5.83 (m, 1H, H-4), 5.08 (dd, 1H, J₁=0.9 Hz, J₂=17.1 Hz, H-5a), 5.02 (dd, J₁=0.8 Hz, J₂=9.7 Hz, H-5b), 2.46 (m, 2H, H-2), 2.38 (m, 2H, H-3); ¹³C-NMR (400 MHz, CDCl₃): 178.6 (q, C-1), 136.5 (t, C-4), 115.8 (s, C-5), 33.4 (s, C:2), 28.7 (s, C-3). The data corresponds to the commercially available compound.

1. A method for performing a chemical reaction to produce a target compound by heating a reaction medium containing a reactant in a reactor, the reaction medium being brought into contact with a solid heating medium that can be heated by electromagnetic induction and that is located inside the reactor and surrounded by the reaction medium and the heating medium being heated by electromagnetic induction with the aid of an inductor, causing the target compound to form, and the target compound being separated from the heating medium, wherein the target compound has a lower molar mass than the reactant and at least one covalent bond of the reactant is cleaved in order to produce said target compound from the reactant.

2. The method according to claim 1, wherein the heating medium is selected from particles of electrically conductive and/or magnetizable solids, the particles having an average particle size in the range from 1 to 1000 nm.

3. The method according to claim 2, wherein the heating medium is selected from particles of electrically conductive and/or magnetizable solids, the particles having an average particle size in the range from 20 to 250 nm.

4. The method according to claim 2, wherein the heating medium is selected from particles of electrically conductive and/or magnetizable solids, each particle containing at least one core of an electrically conductive and/or magnetizable material that is surrounded by a non-magnetic material.

5. The method according to claim 1, wherein the reaction medium is the reactant.

6. The method according to claim 1, wherein it is performed in a continuous-flow reactor that is at least partly filled with the solid heating medium and thus has at least one heating zone that can be heated by electromagnetic induction, the reaction medium flowing through the continuous-flow reactor and the inductor being located outside the reactor.

7. The method according to claim 1, wherein the reactor is designed as a pressure reactor and the chemical reaction is performed at a pressure above atmospheric pressure, preferably of at least 1.5 bar.

8. The method according to claim 6, wherein the reaction medium flows through the continuous-flow reactor one or more times at a speed such that the entire contact time between the reaction medium and the heating medium is in the range from one second to two hours.

9. The method according to claim 1, wherein the reaction medium is present in the reactor as a liquid.

10. The method according to claim 1, wherein the chemical reaction is selected from the group comprising:
a) cleavage or pyrolysis of fats or oils,
b) depolymerization reactions.

11. The method according to claim 10, wherein the chemical reaction is a depolymerization reaction selected from the group comprising: cleavage of polyolefins or of cyanoacrylate polymers or oligomers into smaller fragments or monomers.

12. The method according to claim 1, wherein the inductor generates an alternating field having a frequency in the range from 1 to 100 kHz, preferably in the range from 10 to 80 kHz and in particular up to 50 kHz.

13. The method according to claim 1, wherein the molar mass of the target compound is at most half as large as that of the reactant.
